(19) United States
(12) Patent Application Publication
(10) Pub. No.: US 2017/0128265 A1
(43) Pub. Date: May 11, 2017

(54) VITRECTOR HAVING A ROTATING CUTTER HEAD

(71) Applicant: Carl Zeiss Meditec AG, Jena (DE)

(72) Inventors: Stefan Czaja, Glienicke (DE); Friederike Moll, Aalen (DE); Lisa Rossmann, Aalen (DE)

(21) Appl. No.: 15/344,252

(22) Filed: Nov. 4, 2016

(30) Foreign Application Priority Data
Nov. 10, 2015 (DE) 10 2015 119 378.7

(51) Int. Cl. A61F 9/007

(52) U.S. Cl. CPC A61F 9/00763; A61B 2217/005

(57) ABSTRACT

A device for treating eyes, the device having a lance-like housing which extends along a longitudinal direction (L) and can be inserted into an area of the eye to be treated. The device further includes at least one cutter element, which is arranged at least partially inside the housing and is movable relative to the housing. The housing has an opening through which the cutter element can contact the area of the eye to be treated. The apparatus preferably includes a drive for generating the movement of the cutter element. The cutter element is configured to be rotatable or pivotable with respect to a predefined axis extending in or oblique to the longitudinal direction (L).

### CROSS REFERENCE TO RELATED APPLICATION

This application claims priority of German patent application no. 10 2015 119 378.7, filed Nov. 10, 2015, the entire content of which is incorporated herein by reference.

### FIELD OF THE INVENTION

The present invention relates to a device for treating eyes, in particular to what is called a vitrector. Vitrectomy is understood as the microsurgical removal of a pathologically damaged vitreous body. Operations of this kind are performed, for example, in cases of vitreous haemorrhage that do not resolve, or also in cases of tractional retinal detachment. In these cases, the vitreous body is usually cut out in successive steps and piece by piece using a vitrector. To ensure that the eye does not collapse as the vitreous body is cut out, the intraocular pressure is kept constant with the aid of an infusion. Lighting can be provided by fibre optics. The corresponding treatment elements, which have a diameter of ca. 1 mm, are inserted into the eye in the area of the pars plana. The operating surgeon holds the vitrector in one hand and the light source in the other hand.

### BACKGROUND OF THE INVENTION

Vitrectors known from the prior art have a guillotine-like cutter body inside a housing, which cutter body moves in particular in a longitudinal direction of the housing (up and down) and, via a cutter element, cuts out areas of the vitreous body. Via a suction mechanism, corresponding elements of the vitreous body that have been cut out are aspirated. More recently, vitrectors have also been disclosed that have two cutter elements (moving to and fro along the longitudinal direction), such that a cut can be made during the downward movement and also during the upward movement. A cutting rate of up to 8000 cuts per minute was able to be achieved in this way. However, for improved operations, it would in some cases be desirable to increase these cutting rates still further. In other words, the market is developing in the direction of ever increasing cutting rates. However, despite the abovementioned double cut variant, these rates are still limited by the type of construction.

### SUMMARY OF THE INVENTION

Therefore, it is an object of the present invention to provide a device for treating eyes, in particular a vitrectomy instrument, which further increases the cutting rate compared to devices known from the prior art. According to the invention, this object can, for example, be achieved by an apparatus for treating eyes, the apparatus including: a lance-like housing extending along a longitudinal direction (L) and configured to be inserted into an area of the eye to be treated; at least one cutter element arranged at least partially in the housing and configured to be movable relative to the housing; the housing defining an opening; and, the cutter element being configured to contact the area of the eye to be treated via the opening and to be at least one of rotatable and pivotable with respect to a predefined axis extending in or oblique to the longitudinal direction.

A device according to the invention for treating eyes, in particular a vitrector, has a lance-like (or rod-like) housing which extends along a longitudinal direction and can be inserted into an area of the eye to be treated. In addition, the device has at least one cutter element, which is arranged at least partially inside this housing and is movable relative to the housing. Moreover, the housing has at least one opening through which the cutter element can contact the area of the eye to be treated and is able to cut it. Moreover, the device preferably has a drive for generating the movement of the cutter element relative to the housing, and/or the device is preferably able to be coupled to such a drive for generating the movement of the cutter element relative to the housing. This means that the drive does not necessarily have to be a constituent part of the device.

According to the invention, the cutter element is configured to be rotatable or pivotable with respect to a predefined rotation axis or pivot axis and preferably with respect to a predefined axis extending parallel to the longitudinal direction.

Moreover, it is preferably possible that the cutter element is able to be coupled to a drive mechanically, in order thereby to generate the movement of the cutter element.

In contrast to the approaches known from the prior art, in which cutting movements are executed mainly by a longitudinal movement or a rectilinear movement, it is proposed, within the scope of the present invention, that the cutter elements, for example blades, rotate with respect to a predefined axis. The latter is advantageously an axis that is parallel to the longitudinal direction or is the longitudinal direction itself. However, it would also be conceivable for a rotation to take place about axes that are oblique thereto or also about axes that are perpendicular thereto. Through the provision of a rotational movement (or pivoting movement), the cut count, that is, the number of cuts per unit of time, can in particular also be greatly increased.

The cutter element therefore preferably moves on a circular (or arc-shaped) trajectory.

The cutter element can preferably be configured such that, in order to execute the cutting movement, it cooperates with a portion of the housing. This portion can in particular be an edge that borders the opening. In this connection, it will be noted that the cutter element does not necessarily need to be an element which is already intended per se to cut material. It would also be possible that this cutter element is suitable and intended for cutting in cooperation with other elements, for example the abovementioned edge of the housing.

Advantageously, the housing has an external cross section of between 0.1 mm and 2 mm, preferably between 0.2 mm and 1.5 mm, preferably between 0.2 mm and 1.0 mm and particularly preferably between 0.3 mm and 0.8 mm. These small dimensions allow the device to be inserted in a particularly suitable manner into the eye to be treated and in particular into the vitreous body of the latter. The cutter element is advantageously arranged on a carrier rotatable about the axis. It is particularly preferable that this carrier is fixed in relation to the housing in the longitudinal direction. This means that the cutting movement is in particular a purely rotational movement and not a movement in the longitudinal direction of the housing. The carrier is preferably symmetrical in shape with respect to the rotation axis. It is particularly preferable that the carrier, including the cutter elements arranged thereon, is symmetrical, and in particular rotationally symmetrical, with respect to the rotation axis.

In another advantageous embodiment, the device has a suction unit which serves to at least partially aspirate material cut by the cutter element. The material cut by the cutter element can in particular be organic material of the vitreous body to be treated.

The suction unit can be, for example, a pump mechanism, in particular, but not exclusively, a venturi pump or a peristaltic pump. This pump can be in fluidic communication with the opening of the housing via a channel that extends in particular through the device.

In another advantageous embodiment, the carrier on which the cutter elements are arranged has a tubular shape and/or has a channel, in particular a continuous channel, that in particular also extends in the longitudinal direction. Material from the vitreous body can be aspirated through this channel. In a further configuration, it would be conceivable that a hose element is arranged on an opposite end of the housing, through which hose element the material of the vitreous body can be aspirated (in particular by the pump mechanism). The pump mechanism is preferably spaced apart from the housing. The carrier, on which the cutter elements are arranged, can also be designated as cutter head.

In another advantageous embodiment, the drive is suitable and intended for driving the cutter element and/or the carrier at rotational speeds of at least 2000 revolutions per minute. The drive is advantageously suitable for generating rotational speeds of at least 3000 revolutions per minute, preferably at least 4000 revolutions per minute, and particularly preferably at least 5000 revolutions per minute. In this way, the cutting speed can be greatly increased.

Moreover, the drive can preferably be coupled directly to the carrier on which the cutter elements are arranged. However, the drive is preferably spaced apart from the housing. In an advantageous embodiment, a rotation transmission mechanism is provided which transmits a rotational movement of the drive to the carrier. A coupling mechanism can in this case be provided which couples a rotational movement of the drive to the carrier (on which the at least one cutter element is arranged).

In another advantageous embodiment, the at least one opening is formed in a circumferential wall of the housing. The opening is particularly preferably arranged near the distal end or front end of the housing (that is, the end by which the housing is first inserted into the vitreous body of the eye in an operation). The at least one cutter element is preferably accessible through this opening.

This opening is preferably formed as a slit in the housing. A longitudinal direction of this slit preferably extends parallel to the longitudinal direction of the housing. This permits very precise positioning of the cutter element in the eye to be treated. However, it would also be conceivable that a longitudinal direction of the opening extends perpendicularly with respect to a direction of extent of the cutter element. This would be advantageous, for example, in the case of a spiral configuration of the cutter element.

In another advantageous embodiment, the cutting edge of the cutter element extends at least also in the longitudinal direction of the housing. It is thus possible that this cutting edge extends rectilinearly in the longitudinal direction. However, it would also be possible that the cutting edge extends obliquely with respect to this longitudinal direction. It would also be conceivable that the cutting edge has a spiral configuration, which winds in particular about the rotation axis. With a spiral configuration of this kind, a certain aspirating effect (in the manner of a spiral pump) could already be obtained by the rotation.

In another advantageous embodiment, pushing means can be provided on the carrier, which pushing means convey cut material in the direction of the rotation axis. These could preferably be pushing means which, through the rotational movement of the carrier, convey the cut material in the direction of the rotation axis. Thus, for example, the cutter element could be adjoined by an oblique surface which extends at least also in the direction of the rotation axis of the carrier and which conveys the material in the direction of the rotation axis. In this area of the rotation axis of the carrier, the cut material can then be aspirated (in particular through a channel formed in the carrier).

In another advantageous embodiment, the drive is a pneumatically operated drive. It is thus conceivable that the carrier on which the cutter elements are arranged is set in motion (at least indirectly) by (compressed) air. It is thus proposed that a rotating cutter tube be provided instead of the guillotine, known from the prior art, which moves up and down. In addition, a kind of worm conveyor could also be formed by the spiral configuration of the cutter element. Generally, however, the drive is understood as a mechanism which, optionally with further means, is able to initiate the rotational movement of the cutter element.

Particularly high speeds of rotation and therefore also high cutting rates can be achieved by the pneumatic drive. Overall, this affords higher numbers of cuts and, if appropriate, even an aspiration of the vitreous body. The pneumatic drive can preferably have a turbine, which is driven by a pneumatic medium. This approach also affords advantages in terms of the weight of the vitrector and also in terms of handling, particularly in a sterile area.

It would thus be conceivable that, in contrast to the prior art, no electrical drive unit would have to be set up outside the sterile area. Sterile air, for example, can be used as medium for the pneumatic drive. The device can in this case have a supply line, which supplies sterile air to the drive or to the drive turbine in order to set the latter in rotation. Moreover, a removal line can preferably be provided to remove the sterile air from this drive turbine.

In another advantageous embodiment, at least the housing is made from a metal. The body on which the cutter elements are arranged is also preferably made from a metal. In another advantageous embodiment, the device is at least in part a disposable device. It is thus possible, in particular, that the housing and the cutter element are configured as disposable elements. The cutter elements can be configured as cutting edges and/or blades. Preferably, the drive is not a constituent part of this disposable device. However, the disposable device can in this case have a coupling element for coupling of the drive.

It is advantageous that no bearing elements are provided between the cutter elements and the housing. It is also advantageous that no bearing elements for bearing the cutter elements and the carriers thereof are provided in the area of the opening through which cutting is performed.

It would also be conceivable, moreover, that the housing has a plurality of openings in the circumferential wall.

In another advantageous embodiment, the device has at least two cutter elements arranged on a carrier. These cutter elements are preferably spaced apart from each other in a rotational or circumferential direction. The at least two cutter elements are advantageously spaced uniformly apart from each other. By the provision of two such cutter elements, the cutting rate can in particular also be increased at a constant rotational speed.

At least three cutter elements, particularly preferably at least four cutter elements, are preferably arranged on the carrier. A space or gap is particularly preferably formed between two cutter elements adjacent to each other in a circumferential direction, which space or gap preferably likewise extends in the longitudinal direction of the housing. Cut material can in particular be carried off through this gap.

He drive advantageously effects complete revolutions of the cutter elements. However, it would also be conceivable that the carrier on which the cutter elements are arranged is not configured to make complete revolutions and instead pivots about a defined pivot angle. In this case too, although to some extent a guillotine-like cutting movement would be performed, the cutter elements would nevertheless move along an arc-shaped trajectory.

A recess is particularly preferably provided between at least two cutter elements. In this way, material of the vitreous body, once it has been cut by a cutter element, can be aspirated through a corresponding recess, in particular the abovementioned hollow space of the main body.

### BRIEF DESCRIPTION OF THE DRAWINGS

The invention will now be described with reference to the drawings wherein:

FIG. 1 shows a highly schematic view of a first embodiment of the present invention;

FIG. 2 shows a highly schematic view of a further embodiment of the present invention; and,

FIG. 3 shows a highly schematic view of a further embodiment of a device according to the invention.

### DESCRIPTION OF THE PREFERRED EMBODIMENTS OF THE INVENTION

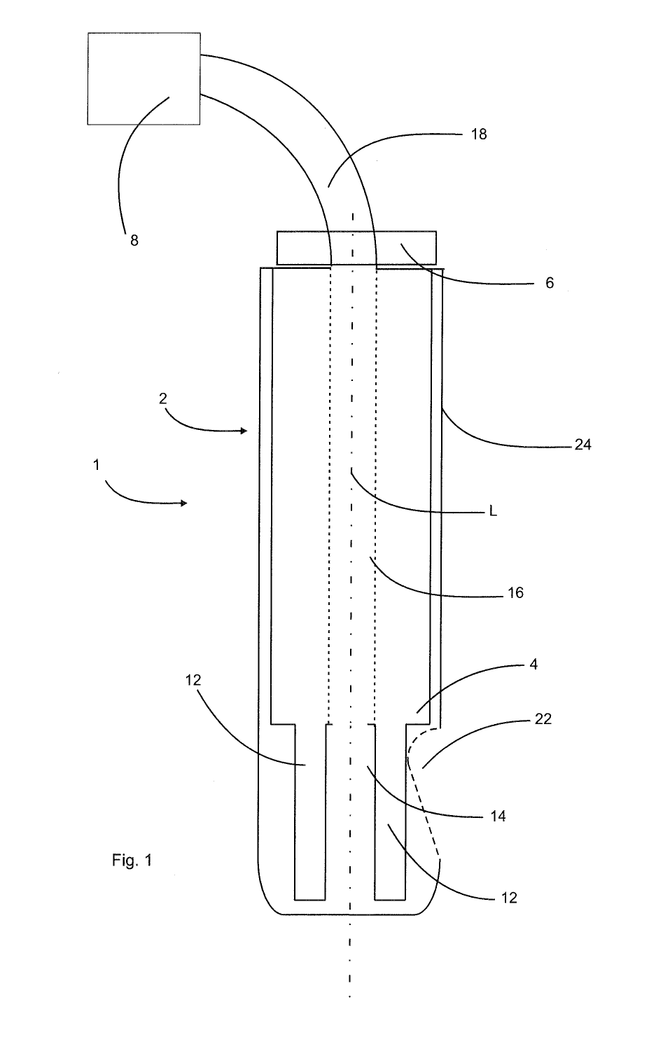

FIG. 1 shows a highly schematic view of a device 1 according to the invention for treating eyes, in particular a cutting/aspirating device. This device has a housing, which is designated overall by reference number 2 and within which a carrier 4 is arranged, on which in turn two cutter elements 12 are arranged. It is possible that these cutter elements are formed integrally with the carrier. The reference sign L designates the longitudinal direction along which both the housing 2 and the cutter elements 12 extend. This longitudinal direction is in particular also the direction in which the device 1 is inserted for treatment into an eye or into the vitreous body of the latter.

The carrier 4 is rotatable with respect to this longitudinal direction. For this purpose, a drive 6 can be provided that generates the rotational movement. However, the drive 6 is preferably not arranged on the housing but instead at a distance therefrom, for example on a central appliance. The rotational movement can be transmitted to the housing and the carrier 4 via a spindle-like body. The cutter elements move along a circular trajectory and, in the present case, preferably in a plane to which the longitudinal direction L is perpendicular. However, it would also be conceivable that the drive is coupled to the housing 2. The longitudinal direction L extends advantageously rectilinearly. Here, the longitudinal direction L also represents the rotation axis with respect to which the carrier 4 rotates.

Thus, during the rotation, the carrier 4 with the cutter elements arranged thereon turns at high speed. However, it would also be conceivable that, instead of a complete rotational movement, a pivoting movement is performed, for example a pivoting movement about a predefined pivoting angle, for example a pivoting angle of 90°.

Reference sign 22 designates an opening through which the eye to be treated can be cut. The vitreous body to be treated can be cut by the cutter elements through the opening 22.

In the interior of the carrier 4 there is a channel 16, which at least also extends along the longitudinal direction L. Cut material from the vitreous body can be aspirated through this channel 16. Reference sign 18 designates a connecting line (in particular a suction line), for example a hose, which also serves to aspirate material from the vitreous body of the eye. Reference sign 8 designates a vacuum generator, for example a peristaltic pump or a vacuum pump, which is suitable and intended for aspirating the material from the eye. It would also be possible for a plurality of openings 22 to be provided in the circumferential direction of the housing 2. Although two cutter elements are shown, it would also be possible for more of these to be arranged on the carrier 4 in order thereby to further increase the cutting rate. Reference sign 24 designates a wall of the housing 2. Except for the opening 22, this wall preferably completely surrounds the carrier 4 in the circumferential direction thereof. The housing preferably has a circular or elliptic cross section. Preferably, this cross section is constant at least in part along the longitudinal direction L.

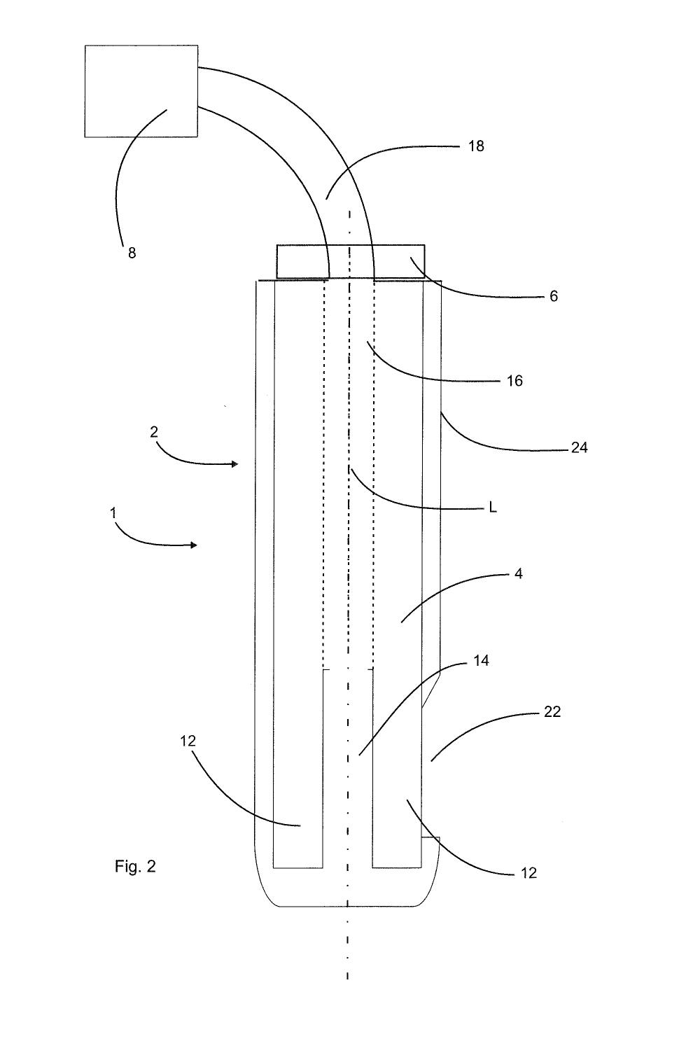

FIG. 2 shows a further embodiment of the device according to the invention. In contrast to the embodiment shown in FIG. 1, the two cutter elements 12 here are set further outwards with respect to the carrier. In this embodiment too, a gap 14 is provided between the cutter elements 12, from which gap the cut material can again be aspirated through the channel 16 and the line 18.

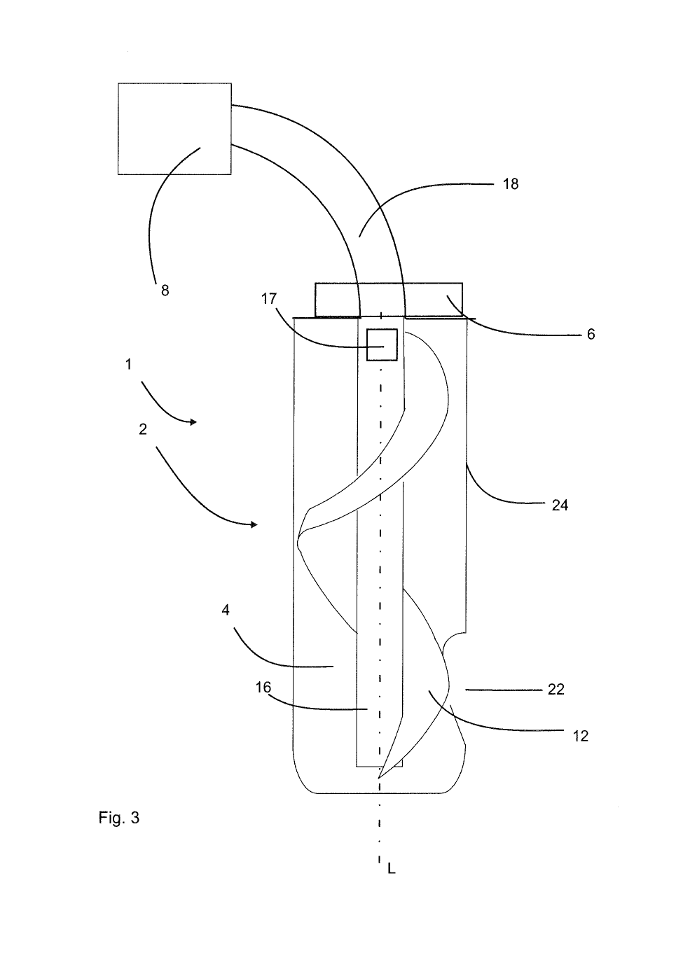

FIG. 3 shows a further embodiment of a device according to the invention. In this embodiment, the cutter element 12 is arranged in a spiral shape on the carrier 4. This carrier can again be a hollow carrier which, in its interior, has a hollow space for aspiration of the cut material. However, in this embodiment of the cutter element, it would also be possible that the cut material is conveyed through the spiral (which for this purpose preferably cooperates with the inner wall of the housing) to an opening 17 of the channel 16.

In this embodiment too, however, it is preferable to provide a line 18, for aspirating the cut material, and also a pump 8. In the embodiment shown here, the carrier 4 will move anticlockwise, as seen from above, such that the cutter element itself also serves as a conveyor element for conveying the material of the eye.

It is understood that the foregoing description is that of the preferred embodiments of the invention and that various changes and modifications may be made thereto without departing from the spirit and scope of the invention as defined in the appended claims.

### LIST OF REFERENCE SIGNS

1 device
2 housing
4 carrier
6 drive
8 vacuum generator, pump
12 cutter element
14 gap
16 channel in carrier 4
17 opening of the channel
18 connecting line
22 opening
24 circumferential wall
L longitudinal direction

What is claimed is:

1. An apparatus for treating eyes, the apparatus comprising:
a lance-like housing extending along a longitudinal direction (L) and configured to be inserted into an area of the eye to be treated;
at least one cutter element arranged at least partially in said housing and configured to be movable relative to said housing;
said housing defining an opening; and,
said cutter element being configured to contact the area of the eye to be treated via said opening and to be at least one of rotatable and pivotable with respect to a predefined axis extending in or oblique to said longitudinal direction.

2. The apparatus of claim 1 further comprising:
a carrier configured to be rotatable about said predefined axis; and,
said cutter element being arranged on said carrier.

3. The apparatus of claim 1 further comprising a suction unit configured for at least partial aspiration of material cut by said cutter element.

4. The apparatus of claim 2, wherein said carrier has a tubular shape.

5. The apparatus of claim 1 further comprising a drive configured to drive said cutter element at rotational speeds of at least 2000 revolutions per minute.

6. The apparatus of claim 2 further comprising a drive configured to drive at least one of said cutter element and said carrier at rotational speeds of at least 2000 revolutions per minute.

7. The apparatus of claim 1, wherein:
said housing has a circumferential wall; and,
said opening is formed in said circumferential wall of said housing.

8. The apparatus of claim 1, wherein said cutter element has a cutter edge extending in said longitudinal direction (L).

9. The apparatus of claim 1 further comprising a pneumatically operated drive configured to drive said cutter element.

10. The apparatus of claim 2 further comprising a pneumatically operated drive configured to drive at least one of said cutter element and said carrier.

11. The apparatus of claim 1 further comprising a drive having a turbine and being configured to drive said cutter element.

12. The apparatus of claim 2 further comprising a drive having a turbine and being configured to drive at least one of said cutter element and said carrier.

13. The apparatus of claim 1 further comprising:
a carrier;
said at least one cutter element including a first cutter element and a second cutter element; and,
said first cutter element and said second cutter element being arranged on said carrier.
